(19) United States
(12) Patent Application Publication
(10) Pub. No.: US 2012/0084873 A1
(43) Pub. Date: Apr. 5, 2012

(54) SEX-DETERMINATION AND METHODS OF SPECIFYING SAME

(76) Inventors: Andrew Sinclair, Victoria (AU); Craig Smith, Victoria (AU); Timothy James Doran, Victoria (AU); Robert John Moore, Victoria (AU); John William Lowenthal, Victoria (AU)

(21) Appl. No.: 13/147,856

(22) PCT Filed: Feb. 8, 2010
(86) PCT No.: PCT/AU2010/000133

§ 371 Date: Dec. 16, 2011

(30) Foreign Application Priority Data
Feb. 8, 2009 (AU) 2009900452

(51) Int. Cl. A01K 67/027; G06Q 90/00; C07H 21/02; A61K 31/7088; C12N 15/85

(52) U.S. Cl. 800/19; 800/8; 800/13; 800/21; 514/44 A; 435/320.1; 536/24.5; 705/500

(57) ABSTRACT

The present invention relates generally to the field of sex determination of animals. Provided are methods and agents to manipulate sex determination, particularly in avian animals such as chickens, through a male chromosome-linked testis (sex) regulatory gene. Expression or activity of the DMRT1 gene or protein is modulated to produce animals with displaying a phenotype sex that differs from their genotype.

### FILING DATA

This application is associated with and claims priority from Australian Provisional Patent Application No. 2009900452, filed on 8 Feb., 2009, entitled “Sex-specified avians and methods of producing same”, the entire contents of which, are incorporated herein by reference.

### FIELD

The present invention relates generally to the field of sex determination. More particularly, the present invention provides methods and agents to manipulate sex determination in species having a male chromosome-linked sex regulatory gene.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Sex is generally chromosomally determined in most animal species (Ellegren, Trends Ecol Evol 15:188-192, 2000; Mizuno et al, Cytogenet Genome Res 99:236-244, 2002). In poultry birds and other avian species, the homogametic sex is male (ZZ) and the heterogametic sex is female (ZW). The mechanism of sex determination in an avian embryo has remained elusive with one hypothesis being that the W (female) chromosome carriers a dominant-active ovary determinant (Arit et al, Proc. Biol. Sci. 271 Suppl. 4:S249-251, 2004; Nakagawa, Trends Genet 20:479-480, 2004). An alternative hypothesis is the dosage of a Z (male)-linked gene wherein two doses (ZZ) determines masculinity (Smith and Sinclair, Bio Essays 26:120-132, 2004).

The ability to be able to determine and manipulate sex determination in a range of species is particularly important in the agricultural industry. To selectively produce female chickens, for example, would facilitate increased economic production of eggs and minimize the unnecessary rearing of male birds. Conversely, male birds are preferred for meat production. By producing single sex lines of chickens it obviates the need for individually sexing every hatchling. It also means that 50% of the hatchlings do not need to be culled because they are not of the required sex. Sex is currently determined in chickens manually by visual inspection but this is time-consuming, tedious and can be inaccurate.

Sex can also be determined by assaying for certain genetic or protein markers. Such methods of sex determination generally require a level of technical expertise and facilities which are not readily available in commercial operations. Furthermore, such methods do not lend themselves to automation, especially in agricultural environments.

There is a need to be able to provide non-human species with a specified sex.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In accordance with the present invention, it is determined that the Z-linked gene, DMRT1, in its homozygous form, confers testis development in avians. Down-regulation of DMRT1 leads to ovarian development in avian embryos. When DMRT1 expression is reduced, affected male embryos exhibit sex reversal, characterized by a feminized left gonad and right testis. The feminized gonad exhibits reduced DMRT1 expression, disorganized testis cords and a decline in testicular biomarkers (e.g. SOX9). Hence, DMRT1 is shown to be a Z chromosome-linked testis (male) regulatory gene and plays a pivotal role in avian male sex determination. The DMRT1 gene has homology in a number of species including fish, reptiles and amphibians. The ability to induce feminization in avians enables efficient production of sex-specified animals as required, such as for use in the agricultural industry.

Accordingly, one aspect of the present invention provides a sex specified animal, the animal or its parent genetically modified to either (i) inhibit expression of DMRT1 or a male chromosome-linked homolog thereof or reduce DMRT1 protein-activity; or (ii) to elevate expression of an DMRT1 or its homolog or elevate DMRT1 protein activity wherein reduced expression of DMRT1 or DMRT1 protein activity leads to an animal with female characteristics and elevated expression of DMRT1 or DMRT1 protein-activity leads to an animal with male characteristics.

By “elevated expression” of DMRT1 means that the level of DMRT1 protein produced are at least equivalent to the expression of two alleles of DMRT1, i.e. in a normal ZZ male. Similarly, a “reduced expression” of DMRT1 means a reduction from the normal ZZ male level. In a particular embodiment, the animal is an avian animal.

Accordingly, another aspect of the present invention provides a sex-specified avian animal, the avian animal or its parent genetically modified to change the level of expression of DMRT1 or its homolog or change the level of activity of DMRT1 protein or its homolog, which level of DMRT1 expression or DMRT1 protein activity determines the sex of the avian animal.

More particularly, the present invention provides a sex-specified avian animal, the avian animal or its parent genetically modified to minimise a modified level of expression of DMRT1 or its homolog or minimize the level of activity of DMRT1 protein or its homolog, wherein a reduced level of DMRT1 expression or DMRT1 protein activity leads to an avian animal with female characteristics.

A further aspect of the present invention provides a sex-specified avian animal, the avian animal or its parent genetically modified to elevate the level of expression of DMRT1 or its homolog or elevate the level of activity of DMRT1 protein or its homolog wherein elevated expression of DMRT1 or of DMRT1 protein activity leads to an avian animal with male characteristics.

As indicated above, in terms of avian species such as chickens, an elevated level of DMRT1 means to a level similar to a normal (ZZ) male. A reduced level means a similar to a normal (ZW) female.

Another aspect of the present invention is directed to a method for generating a sex-specified animal, the method comprising introducing into a blastoderm or developing embryo of the animal an agent which modulates the level of expression of DMRT1 or a male chromosome-linked homolog thereof or modulates the activity of DMRT1 protein and allowing the embryo to develop into a postnatal animal wherein an agent which reduces expression of DMRT1 or DMRT1 protein activity results in an animal with female characteristics and an agent which elevates expression of DMRT1 or DMRT1 protein activity results in an animal with male characteristics.

In a particular embodiment, the animal is an avian animal.

Consequently, another aspect of the present invention provides a method for generating a sex-specific avian animal, the method comprising introducing into a blastoderm or developing embryo of the avian animal an agent which modulates the level of expression of DMRT1 or its homolog or the level of activity of DMRT1 protein, allowing the embryo to develop to a hatchling wherein the hatchling having female characteristics comprises a reduced expression of DMRT1 or reduced DMRT1 protein activity and a hatchling having male characteristics comprising elevated expression of DMRT1 or elevated DMRT1 protein-activity.

Still another aspect of the present invention is directed to a method for generating a sex-specified avian animal, the method comprising modulating the level of expression of DMRT1 or its homolog or the activity of DMRT1 protein in the avian animal for a time and under conditions sufficient to decrease DMRT1 expression to generate a female avian animal or to induce DMRT1 expression to generate a male avian animal.

Yet another aspect of the present invention contemplates a method of inducing feminization of an avian embryo, the method comprising introducing to the embryo an agent which reduces the functional level of DMRT1 expression or DMRT1 protein activity for a time and under conditions sufficient for the embryo to develop female characteristics.

Still yet another aspect of the present invention contemplates a method of inducing masculization of an avian embryo, the method comprising introducing to the embryo an agent which comprises DMRT1 protein or its functional homolog or analog or which facilitates expression of DMRT1 or its functional homolog for a time and under conditions sufficient for the embryo to develop male characteristics.

In an embodiment, the agents of the present invention are genetic constructs or compounds which either down-regulate expression of an endogenous DMRT1 gene or within elevated expression of DMRT1. Examples include viral vectors, microRNAs (miRNA), antisense oligonucleotides or compounds, RNAi molecules, siRNA molecules, sense oligonucleotides, ribozymes, dsRNA molecules and oligonucleotides comprising hairpin loops. Such molecules are proposed to target DMRT1 expression and to reduce levels of DMRT1 protein. In another embodiment, the agents are genetic constructs which, when expressed or introduced into the avian chromosome, produce DMRT1 protein. In yet another embodiment, the agents are chemical molecules which inhibit the function of DMRT1 protein or which inhibit the expression of the DMRT1 gene. Still, in another embodiment, DMRT1 protein or DNA or RNA is injected into the embryo to induce mascularization of an avian embryo.

The present invention further contemplates genetically modified fertilized avian eggs, the eggs comprising a blastoderm or developing embryo genetically modified to either reduce expression of DMRT1 to a level less than a homozygous DMTR1 male or to elevate expression of DMRT1 to the level of a homozygous DMRT1 male. Such eggs are then provided to a consumer on the basis that they will substantially give rise to all female or all male hatchlings.

Reference to avian species or animals herein includes inter alia chickens, ducks, geese, turkeys, bantams, pheasants and quail. However, the present invention extends to any avian species including penguins, aviary birds, game birds, bird pests and the like. The present invention further extends to non-human animals other than avian species such as fish, reptiles and amphibians.

Reference to “sex” includes gender.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

TABLE 1
List of Sequences

| SEQ ID NO: | DESCRIPTION |
|---|---|
| 1 | DMRT1 probe (forward) |
| 2 | DMRT1 probe (reverse) |
| 3 | HPRT probe (forward) |
| 4 | HPRT probe (reverse) |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a photographic and diagrammatic representation of knockdown of DMRT1 protein in vitro, using RCASBP virus to deliver miRNA against DMRT1. Immunofluorescent detection of DMRT1 protein, and GFP fluorescence. (a) Chicken fibroblastic DF1 cells infected with RCASBP.A.DMRT1 only, showing no GFP expression but robust over-expression of DMRT1 protein in cell nucleic (red). (b) Cells infected with RCASBP.A.DMRT1 after infection with virus carrying a non-silencing control scrambled sequence with GFP reporter (scrambled control), showing widespread GFP and DMRT1 protein expression (i.e. no knockdown). In the merged image, some cells appear yellow or orange, because the GFP can have both cytoplasmic and nuclear localization. (c) Cells infected with RCASBP.A.DMRT1 following infection with virus carrying DMRT1 miRNA, showing widespread expression of GFP (green) and knockdown of DMRT1 protein (red). Note that a few cells lacking GFP expression (and hence no miRNA) show strong DMRT1 expression (arrow). (d) Knockdown of DMRT1 mRNA expression in DF1 cells treated with RCASBP.B.GFP.DMRT1 compared to cells treated with RCASBP.B.scrambled control Uninfected DF1 cells show no endogenous DMRT1 expression.

FIG. 2 is a photographic representation of abnormal gonadal development in male gonads treated with DMRT1 miRNA. Urogenital systems from day 10 chicken embryos treated at day 0 with scrambled control or DMRT1 miRNA. Gonads are outlined for clarity. (a) Control male (ZZ), showing bilateral development of symmetrical testes. (b) Control female (ZW), showing typical asymmetric development, characterized by larger left ovary and smaller right gonad. (c) Genetic male (ZZ) infected with virus carrying DMRT1 miRNA, showing abnormal (female-like) asymmetry, with a larger left and smaller right gonad. (d) Female (ZW) infected with virus carrying DMRT1 miRNA, showing normal asymmetric development. Ms-paired mesonephric kidneys.

FIG. 3 is a photographic representation of feminization of genetic male (ZZ) chicken embryos treated with DMRT1 miRNA. (a) Normal expression of DMRT1 protein in a 10 day old genetic male embryo (ZZ) treated with non-silencing scrambled control sequence. DMRT1 is strongly expressed in the Sertoli and germ cells of the organizing testis cords (arrows). (b) Internal distribution of germ cells within the testis cords of a control male, as assessed by staining for Chick Vasa Homologue (CVH). (Still yet another aspect of the present invention contemplates a method of inducing masculization of an avian embryo, the method comprising introducing to the embryo an agent which comprises DMRT1 protein or its functional, homolog or analog or which facilitates expression of DMRT1 protein or its functional homolog for a time and under conditions sufficient for the embryo to develop male characteristics. (c) Widespread expression of GFP in a control male gonad. (d) Reduced DMRT1 protein and disorganized cords in a left male (ZZ) gonad treated with DMRT1 miRNA. Compare with (a) above. Some areas show normal DMRT1 expression (e.g. arrows), but expression is weak in other areas and cord formation is lost. (e) Cortically biased (female-like) germ cell distribution in a male gonad treated with DMRT1 miRNA (e.g. arrows). (f) Widespread GFP expression in a gonad treated with DMRT1 miRNA, characterized by normal DMRT1 expression in a small right testis and greatly reduced DMRT1 expression in a left ovary. (h) Female-like germ cell distribution in the left ovary shown in (g). (i) GFP expression in the left ovary shown in (h). (j) Left ovary of a control female (ZW), showing low DMRT1 expression throughout the gonad, except for high level expression in cortical germ cells. (k) Typical cortical germ cell distribution in left ovary of control female, as assessed by CVH. (l) Widespread GFP expression in a control female gonad.

FIG. 4 is a photographical representation of down-regulation of male markers and activation of female markers in male gonads treated with DMRT1 miRNA. (a) Lack of SOX9 expression in a ZW control female. (b) Normal SOX9 expression in organized testis cords of a control male (ZZ) treated with scrambled control miRNA sequences. (c) Reduced SOX9 expression and disorganized testis cords in a ZZ male treated with DMRT1 miRNA. (d) Normal Aromatase enzyme expression in the paired gonads of a control female (ZW) treated with scrambled miRNA. (c) Lack of aromatase expression in the (left) gonad of a control male (ZZ) treated with scrambled miRNA sequence. (f) Ectopic activation of aromatase in the left gonad of a ZZ male treated with DMRT1 miRNA. (g) Higher power view of aromatase expression in the left gonad of a control female (ZW), showing expression in cells surrounding the characteristic lacunae (cavities) of the medulla (arrows). (h) Partial DMRT1 expression in a ZZ male treated with DMRT1 miRNA. DMRT1 expression is reduced and testis cords are disrupted in the bracketed area, which now ectopically expresses aromatase, shown in (i). The region expression aromatase shows female-like lacunae (e.g. arrowheads).

FIG. 5 is a diagrammatic representation of the design of shuttle plasmid for cloning cGFP.shRNA into RCASBP.B viral vector, Plasmid pRmiR (RCAS miRNA) is derived from pcDNA6.2-GW/EmGFP-miR (Invitrogen). Three ds-oligos were cloned into the parent vector to allow for cloning of siRNA ds-oligos into the miRNA cassette (via the back to back BsaI sites) and provision of two Cla1 sits up- and down-stream of the eGFP/miR cassette to allow cloning into RCAS. Once cloned into RCAS, a single polyadenylated cGFP/miRNA transcript is produced from the viral LTR resulting in expression of eGFP protein and mature siRNA within the same cell.

### DETAILED DESCRIPTION

In accordance with the present invention, sex determination in avian species is specified by expression of the Z-linked gene, DMRT1 to produce DMRT1 protein. The DMRT1 gene is described by Shan et al, Cytogenetics Cell Genetics 89:252-257, 2000; Smith et al, Nature 402:601-602, 1999. It is proposed herein that dosage dependent (i.e. two alleles in a homozygous [ZZ] male) expression of DMRT1 leads to masculinization of an avian embryo. Similarly, down-regulating levels of DMRT1 expression or DMRT1 protein leads to feminization of an avian embryo.

DMRT1 is, therefore, regarded as a male chromosome-linked sex regulatory gene. In generic terms, if M is considered a male gamete and F is considered a female gamete, a homogametic sex (MM) is male and the heterogametic sex (MF) is female. It is proposed that an M chromosome-linked homolog of DMRT1 confers masculinity in the homogametic state. Hence, a dose of DMRT1 conferred by an MM male is considered here as a 2x dose.

A female animal (MF) may exhibit a 1x dose. Hence, it is proposed herein that feminization occurs by manipulating a MM chromosome to exhibit less than a 2x (<2x) dose of DMRT1 or its M-linked homolog. By “less than 2x” or “<2x” includes meant from 0x to 1.5x which encompasses amounts such as 0, 0.1, 0.2, 0.3, 0.4, 0.5, 0.6, 0.7, 0.8, 0.9, 1.0, 1.1, 1.2, 1.3, 1.4 and 1.5x, or amounts inbetween.

Hence, the present invention contemplates targeting DMRT1 expression or DMRT1 protein activity or a male chromosome-linked homolog thereof in order to manipulate and specify animal gender. Reduced expression of DMRT1 or its male chromosome-linked homolog to less than the level of expression of two alleles in a normal male results in feminization. Elevated expression of DMRT1 or its male chromosome-linked homolog to the level of expression of two alleles in a normal male results in mascularization.

Genetic, proteomic and chemical approaches are therefore contemplated herein to either reduce levels of DMRT1 expression or DMRT1 protein activity in order to generate female animals or to induce DMRT1 expression or DMRT1 protein production to favor generation of male animals.

Accordingly, one aspect of the present invention provides a sex specified animal, the animal or its parent genetically modified to either (i) inhibit expression of DMRT1 or a male chromosome-linked homolog thereof or reduce DMRT1 protein-activity; or (ii) to elevate expression of an exogenous DMRT1 or its homolog or elevate DMRT1 protein activity wherein reduced expression of DMRT1 or DMRT1 protein activity leads to an animal with female characteristics and elevated expression of DMRT1 or DMRT1 protein-activity leads to an animal with male characteristics.

By “elevated expression” of DMRT1 means that the level of DMRT1 protein produced is at least equivalent to the expression of two alleles of DMRT1, i.e. in a normal DMRT1 male (i.e. 2zx dose). Similarly, a “reduced expression” of DMRT1 means less than that of a normal male (i.e. <2x dose). In a particular embodiment, the animal is an avian animal.

The present invention provides, therefore, a gender specified non-human animal and a method of producing same wherein the sex of the animal is determined by the dose (x) of a male chromosome (M)-linked sex regulatory gene and wherein a homogametic (MM) male has a 2x dose of the regulatory gene and a heterogametic (MF) female, wherein F is the female gamete, has a 1x dose of the regulatory gene, the gender specified animal or its parent genetically modified by a means selected from:

(i) down-regulating expression of one or both alleles of the sex regulatory gene on the M chromosome to a dose of <2x, wherein the gender is female;

(ii) up-regulating expression of the sex regulatory gene on a M chromosome in a MF female to a dose level of 2x or more wherein the gender is male; and

(iii) introducing and expressing an exogenous sex regulatory gene to the F or M chromosome, wherein the gender is male;

wherein the sex regulatory gene is DMRT1 or a male chromosome-linked homolog thereof.

Another aspect of the present invention is directed to a method for generating a sex-specified animal, the method comprising introducing into a blastoderm or developing embryo of the animal an agent which modulates the level of expression of DMRT1 or a male chromosome-linked homolog thereof or modulates the activity of DMRT1 protein and allowing the embryo to develop into a postnatal animal wherein an agent which reduces expression of DMRT1 or DMRT1 protein activity results in an animal with female characteristics and an agent which elevates expression of DMRT1 or DMRT1 protein activity results in an animal with male characteristics.

In a particular embodiment, the animal is an avian animal.

Consequently, another aspect of the present invention provides a method for generating a sex-specific avian animal, the method comprising introducing into a blastoderm or developing embryo of the avian animal an agent which modulates the level of expression of DMRT1 or its homolog or the level of activity of DMRT1 protein, allowing the embryo to develop to a hatchling having female characteristics comprises a reduced expression of DMRT1 or reduced DMRT1 protein activity and a hatchling having male characteristics comprising elevated expression of DMRT1 or elevated DMRT1 protein-activity.

Accordingly, another aspect of the present invention provides a sex-specified avian animal, the avian animal or its parent genetically modified to change the level of expression of DMRT1 or its homolog or change the level of activity of DMRT1 protein or its homolog, which level of DMRT1 expression or DMRT1 protein activity determines the sex of the avian animal.

Whilst the present invention extends to any non-human animal having a male chromosome-linked DMRT1 homolog such as fish, reptiles and amphibians, it is particularly directed to avian animals.

More particularly, the present invention provides a sex-specified avian animal, the avian animal or its parent genetically modified to minimise a modified level of expression of DMRT1 or its homolog or minimize the level of activity of DMRT1 protein or its homolog, wherein a reduced level of DMRT1 expression or DMRT1 protein activity leads to an avian animal with female characteristics.

A further aspect of the present invention provides a sex-specified avian animal, the avian animal or its parent genetically modified to elevate the level of expression of DMRT1 or its homolog or elevate the level of activity of DMRT1 protein or its homolog wherein elevated expression of DMRT1 or of DMRT1 protein activity leads to an avian animal with male characteristics. In this regard, an endogenous DMRT1 on a heterogametic female may be manipulated to enhance its expression to a 2x dose amount or a further copy introduced to increase copy number. An “introduced” DMRT1 is referred to as an “exogenous DMRT1”.

Still another aspect of the present invention is directed to a method for generating a sex-specified avian animal, the method comprising modulating the level of expression of DMRT1 or its homolog or the activity of DMRT1 protein in the avian animal for a time and under conditions sufficient to decrease DMRT1 expression to generate a female avian animal or to induce DMRT1 expression to generate a male avian animal.

Yet another aspect of the present invention contemplates a method of inducing feminization of an avian embryo, the method comprising introducing to the embryo an agent which reduces the functional level of DMRT1 expression or DMRT1 protein for a time and under conditions sufficient for the embryo to develop female characteristics.

Still yet another aspect of the present invention contemplates a method of inducing masculization of an avian embryo, the method comprising introducing to the embryo an agent which comprises DMRT1 protein or its functional, homolog or analog or which facilitates expression of DMRT1 or its functional homolog for a time and under conditions sufficient for the embryo to develop male characteristics.

In one embodiment, the agent is a genetic molecule which inhibits DMRT1 expression or which facilitates the generation of a DMRT1 deletion or inactivation such as by introducing a stop codon or insertion.

Examples of such molecules include micro(mi)RNAs, RNAi, siRNA, dsRNA, oligonucleotides comprising hairpin loops, antisense oligonucleotides, sense oligonucleotides or their chemically modified forms including antagomers. In particular, the disruption of the DMRT1 gene may be transient or rendered permanent. In a particular aspect, avian animals are generated with an inability to express DMRT1.

Hence, another aspect of the present invention provides a genetically modified avian animal, the avian animal substantially incapable of expressing DMRT1 or its functional homolog or progeny of the avian animal.

Such an avian animal would exhibit female characteristics and be able to lay eggs.

Consequently, this aspect of the present invention provides a genetically modified female egg laying avian animal, the avian animal substantially incapable of expression DMRT1 or its functional homolog or progeny of the avian animal.

In an embodiment of the present invention, the DNA encoding the endogenous DMRT1 gene in an avian is deleted. Methods for deleting an endogenous gene such as DMRT1 in avians are well known to a person skilled in the art and generally comprise inserting a genetic construct into a pluripotent cell and transferring the cell into an embryo to yield a chimera. Through breeding, the construct becomes integrated into the germline of a resulting animal and ultimately results in the disruption of the production of endogenous DMRT1. The disruption of endogenous DMRT1 production may occur by targeted disruption of a specific DMRT1 gene locus, the substantial deletion of a DMRT1 gene locus, or the insertion of an engineered construct (e.g. stop codon) that, through ordinary processes of cell division, replaces an intact endogenous locus in an embryonic stem cell or in the resulting animal. The construct may also induce gene silencing by, for example, miRNA, RNAi, siRNA and the like. Methods for inactivating genes in avians are further described in, for example, International Patent Publication No. WO 03/081992.

Other mechanisms for silencing DMRT1 expression include gene silencing through epigenetic processes such methylation of all or part of the DMRT1 genetic locus. As indicated above, gene silencing may be induced in any number of ways including the use of miRNA, RNAi, siRNA, siRNA, Zinc-finger nucleases and various dicer-comprising constructs. Hence, the present invention provides an oligonucleotide compound which selectively inhibits expression of an endogenous DMRT1 gene or all or part of the DMRT1 genetic locus. Generally, the inhibition of expression is permanent and is passed on to future generations. However, transient inhibition is also contemplated herein. By “transient” includes days, weeks, months or for the life of the avian animal.

Hence, the present invention employs compounds, preferably oligonucleotides and similar species for use in modulating the function or effect of DMRT1 in animal species. This is accomplished by providing oligonucleotides which specifically target DNA or RNA encoding DMRT1. As used herein, the terms “target nucleic acid” and “nucleic acid molecule encoding DMRT1” have been used for convenience to encompass DNA encoding DMRT1, RNA (including pre-mRNA and mRNA or portions thereof) transcribed from such DNA, and also cDNA derived from such RNA. This aspect encompasses antisense and sense-suppression of DMRT1.

The functions of DNA to be interfered with can include replication and transcription. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One particular result of such interference with target nucleic acid function is a reduction in expression of DMRT1.

It is understood in the art that the sequence of a sense or antisense oligonucleotide compound need not be 100% complementary to that of its target nucleic acid to be effective in inducing inhibition. Moreover, an oligonucleotide may comprise at least 70% sequence complementarity to a target region within the target nucleic acid, more particularly comprise 90% sequence complementarity and even more particularly comprise 95% sequence complementarity to the target region within the target nucleic acid sequence to which it is targeted.

DMRT1 levels may also be reduced functionally by the introduction of an inhibitor of DMRT1 activity or DMRT1 expression. Generally, the inhibitor is produced by genetic means introduced into the avian animal.

Hence, another aspect of the present invention contemplates a genetically modified animal including an avian animal, the animal genetically modified to express genetic material which encodes an inhibitor of DMRT1 activity or DMRT1 expression or of a male chromosome-linked homolog thereof.

Accordingly, the inhibitor may be a protein inhibitor of DMRT1 activity or may be an antisense or sense oligonucleotide which down-regulates DMRT1 expression.

Examples of protein inhibitors include antibody chains, marine animal-derived single chain antibodies (IgNARs) and a peptide inhibitor of DMRT1.

In another embodiment, DMRT1 activity is introduced or enhanced to ensure the generation of male animals including avian animals. Generally, this is accomplished by introducing genetic material which encodes a DMRT1 protein or its functional homolog. Generally, an introduced DMRT1 gene is referred to as an “exogenous” DMRT1. This also still applies to subsequent progeny.

Accordingly, another aspect of the present invention provides a genetically modified animal including an avian animal, the animal genetically modified to express a DMRT1 protein or its homolog or an enhancer of DMRT1 expression or of a male chromosome-linked homolog.

In a further embodiment, genetically modified animals including avian animals are produced which have been genetically modified to enable the DMRT1 gene or gene locus to be selectively disrupted, generally in ovo. For example, the DMRT1 gene or genetic locus may be inducibly inactivated upon certain conditions. Hence, if a female animal is required, inducible inactivation occurs to inhibit DMRT1 expression. However, if a male animal is required, the embryo is not subject to conditions resulting in inactivation of the DMRT1 gene.

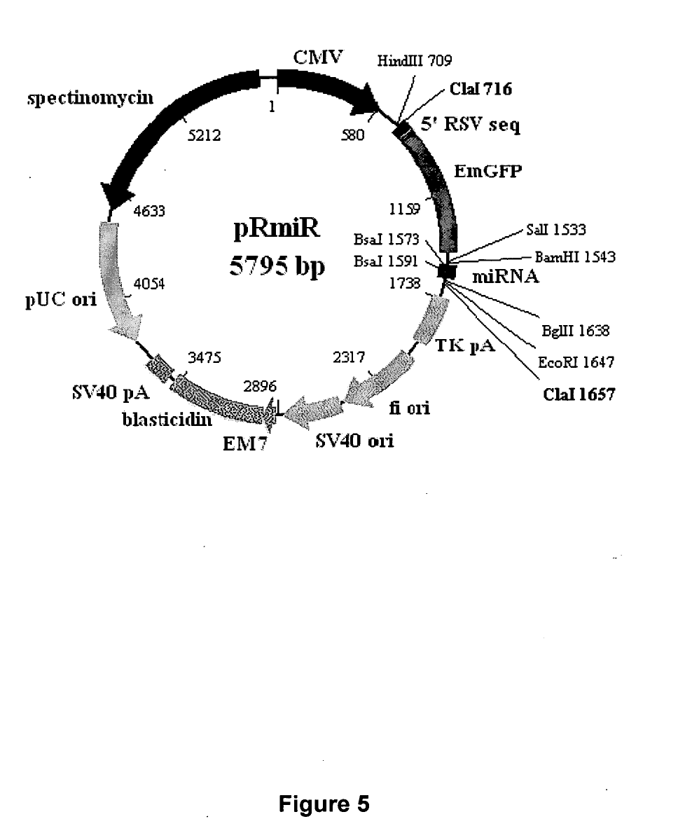

A useful vector to genetically manipulate embryos, is depicted in FIG. 5. Once either DMRT1 expression is disrupted or induced, the level of expression can be determined.

There are many methods which may be used to detect a DMRT1 expression including determining the presence to DMRT1 mRNA via sequence identification. Direct nucleotide sequencing, either manual sequencing or automated fluorescent sequencing can detect the presence of a particular mRNA species.

Techniques for detecting nucleic acid species include PCR or other amplification techniques.

Nucleic acid analysis via microchip technology is also applicable to the present invention. In this technique, distinct oligonucleotide probes are built up in an array on a silicon chip. Nucleic acids to be analyzed are fluorescently labeled and hybridized to the probes on the chip. It is also possible to study nucleic acid-protein interactions using these nucleic acid microchips. Using this technique, one can determine the presence of DMRT1 mRNA species or the level of mRNA as well as the expression of levels of DMRT1.

Hence, alteration of mRNA expression from a DMRT1 genetic locus can be detected by any techniques known in the art. These include Northern blot analysis, PCR amplification and RNase protection. Diminished mRNA expression indicates an alteration of an affected gene. Alteration of DMRT1 expression can also be detected by screening for alteration of expression product such as a protein. For example, monoclonal antibodies immunoreactive with a DMRT1 protein can be used to screen a tissue. Lack of cognate antigen or a reduction in the levels of antigen would indicate a reduction in expression of DMRT1 or a male chromosome-linked homolog thereof. Such immunological assays can be done in any convenient formats known in the art. These include Western blots, immunohistochemical assays and ELISA assays. Any means for detecting an altered protein can be used to detect alteration of the wild-type protein. Functional assays, such as protein binding determinations, can be used.

Hence, the present invention further extends to a method for identifying a genetic basis behind sex in an animal, the method comprising obtaining a biological sample from the animal and detecting the level of expression of DMRT1, wherein the presence of a reduced level of DMRT1 expression of its homolog is instructive as to a female or male animal, respectively.

The biological sample is any fluid or cell or tissue in which DMRT1 is expressed. In one embodiment, the biological sample includes blood, lymph, urine and saliva or cells from these samples.

Consequently, the present invention provides genetic, chemical and proteinaceous agents which either inhibit DMRT1 gene expression or DMRT1 protein activity or which facilitate DMRT1 activity.

Another aspect of the present invention is directed to the use of an agent which inhibits DMRT1 gene expression or DMRT1 protein activity or a male chromosome-linked homolog thereof in the manufacture of a feminized animal, such as an avian animal.

One such agent is the vector in FIG. 5.

Still another aspect of the present invention contemplates the use of an agent which facilitates DMRT1 expression or protein activity or a male chromosome-linked homolog thereof in the manufacture of a mascularized animal such as an avian animal.

Reference to the “DMRT1” gene includes the DMRT1 genetic locus and further includes functional variants (e.g. polymorphic variants) and functional male chromosome-linked homologs thereof. A functional variant or homolog includes a gene having at least 80% identity to the cDNA sequence encoding DMRT1. By at least 80% identity means at least 80, 81, 82, 83, 84, 85, 86, 87, 88, 89, 90, 91, 92, 93, 94, 95, 96, 97, 98, 99 or 100% identity. This can be conveniently determined by any number of algorithmic means. Determination of identity is generally after optimal alignment. Similarly, a functional variant or homolog of DMRT1 includes genes having a DNA strand which hybridizes under medium to high stringency conditions to a strand of DMRT1 DNA.

Reference herein to hybridization includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6x SSC buffer, 0.1% w/v SDS at 25°-42° C.; a moderate stringency is 2x SSC buffer, 0.1% w/v SDS at a temperature in the range 20° C. to 65° C.; high stringency is 0.1x SSC buffer, 0.1% w/v SDS at a temperature of at least 65° C.

Similarly, reference to the DMRT1 protein, includes all functional variants and homologs thereof such as proteins having at least 80% amino acid similarity to DMRT1 after optimal alignment.

Another aspect of the present invention contemplates a business model, such as for the poultry industry. In the business model, poultry birds are genetically modified to inactivate expression of DMRT1. Fertile eggs produced by such poultry birds will give rise to female birds only and hence the level of loss due to unnecessary rearing of male birds substantially reduced.

Hence, a business model is provided for the generation of female poultry birds with enhanced economic returns, the model comprising generating female poultry birds with an inactivated DMRT1 gene or gene locus, mating these birds to one or more male birds to generate fertilized eggs, allowing the eggs to hatch wherein the resulting hatchlings are all female and wherein the female birds are reared and introduced in an existing poultry bird operation.

Such a business model can result in enhanced economic gains by reducing the number of male birds which need to be reared and increasing the number of birds which can, for example, be used for egg production or meat production.

Reference herein to an “avian animal” or “avian species” includes any member of the Class Ayes including poultry birds such as chickens, ducks, geese, turkeys, bantams, pheasants and quail. The methods of the present invention are also applicable to game birds, aviary birds and as a means to control avian animals regarded as pests. As indicated above, the present invention extends to non-human animals other than avian species such as fish, reptiles and amphibians.

The present invention further contemplates genetically modified fertilized avian eggs, the eggs comprising a blastoderm or developing embryo genetically modified to either reduce expression of DMRT1 to a level less than a homozygous DMRT1 male or to elevate expression of DMRT1 to the level of a homozygous DMRT1 male. Such eggs are then provided to a consumer on the basis that they will substantially give rise to all female or all male hatchlings.

Again, the present invention provides a business model to enhance economic returns for the poultry industry, the model comprising supplying genetically modified fertilized eggs with an indication whether the eggs will give rise to female hatchlings or male hatchlings, each egg comprising a genetically modified blastoderm or developing embryo in which either expression of DMRT1 is reduced to a level less than a normal ZZ male or expression of DMRT1 is elevated to the level of a normal ZZ male.

As indicated above, reduced expression of DMRT1 means at a level wherein the embryo will develop female characteristics. An elevation of expression of DMRT1 means to a level wherein the embryo exhibits male characteristics.

The present invention further contemplates genetic elements such as a retroviral vector for use in inducing reduction or elevation in expression of DMRT1 or its male chromosome-linked homolog.

The present invention extends to all progeny of genetically modified animals and to stem cell lines therefrom. Genetically modified hatchlings are also contemplated herein.

The present invention is further described by the following non-limiting Examples. Aspects of the present invention have been published in Smith et al, Nature Letters 46:267-271, 2009, the contents of which, are incorporated herein by reference. In the Examples, the following materials and methods were employed.

### Materials and Methods

### Preparation of Viruses:

RCASBP.B virus was modified to carry the open reading frame of GFP together with a recombinant microRNA directed against chicken DMRT1 in its 3′ UTR. The sequence 5 was designed using the Invitrogen Block-It (Trade Mark) system, and generated in a short hairpin format. The sequence was directed against exon three of chicken DMRT1 mRNA, and did not show significant homology to other sequences in the chicken genome (equal to or greater than 16/21 bases). The hairpin sequence was cloned into an engineered shuttle plasmid carrying GFP (pRmiR—FIG. 5). The GFP-microRNA was then subcloned into RCASBP.B strain virus. High quality DNA was then used to transfect chicken DF1 cells. Virus was harvested from DF1 cells and purified as described previously (Smith et al, Int. J. Dev. Biol. 53:59-67, 2009). Viral titres of approximately 108 infectious units/mL were obtained. For controls, a scrambled sequence of the same bases was used, cloned into RCASBP.B and propagated in the same way (=RCASBP.B.GFP.scrambled control).

### Knockdown of DMRT1 Expression In Vitro:

To test the efficacy of knockdown, DF1 cells were infected with RCASBP.B.GFP.DMRT1 or scrambled control virus, grown for several days, and then infected with RCASBP.A. strain virus carrying DMRT1 open reading frame. (DF1 cells do not produce endogenous DMRT1. Cells can be infected with two viruses provided they are of different strains). After fours days of co-infection, cells were processed for GFP and DMRT1 protein expression. Immunofluorescence was employed, using a DMRT1 antibody (in-house) and Alexfluor secondary antibodies, as described previously (Smith et al, Biol Reprod 68:560-567, 2003). Knock down was also assessed by quantitative real time PCR. RNA was extracted from the cells and cDNA synthesized as previously described (Smith et al, BMC Dev Biol 8:72, 2008). Probes were designed using the UPL Assay Design Centre (https://www.roche-applied-science.com) and are as follows; DMRT1 probe #59 For 5′-AGCCTCCCAGCAACATACAT-3′ (SEQ ID NO:1) and rev 5′-GCGGTTCTCCATCCCTTT-3′ (SEQ ID NO:2); HPRT probe #38 For 5′-CGCCCTCGACTACAATGAATA-3′ (SEQ ID NO:3), Rev 5′-CAACTGTGCTTTCATGCTTTG-3 (SEQ ID NO:4)′. Analysis was performed using LightCycler 480 instrument and software (Roche).

### Embryos:

Fertile chicken eggs (Gallus gallus domesticus) were obtained from SPAFAS, Woodend, Victoria, Australia. Four hundred day 0 blastoderms were injected with DMRT1 knockdown or scrambled control virus using a fine glass capillary needle. Eggs were sealed and incubated until day 10. Survival to day 10 was 40%. Embryos showing 10 GFP fluorescence in the urogenital system (28/160) were selected for further analysis. GFP expression in the urogenital system varied from strong to weak. Embryos were genotypically sexed by PCR as described previously (Smith et al, 2003, supra). Of the 28 embryos, five males that showed strong GFP in the gonads were found to have varying degrees of DMRT1 knockdown and evidence of feminisation. Several females showed strong GFP 15 in the gonads but normal ovarian development. All other embryos (15) had variable GFP in the gonads, normal gonadal sex differentiation and no DMRT1 knockdown. It as hypothesized that lower levels of microRNA delivery (as assessed by lower GFP expression) may be insufficient to influence endogenous DMRT1. (There was a good inverse correlation between GFP expression level and DMRT1 expression; embryos 20 with stronger GFP showed stronger knockdown of DMRT1, as anticipated). All genetic male embryos that showed feminization were re-sexed, using tissue derived directly form the urogenital system. In all cases, the originally assigned genotypic sex was confirmed.

### Immunofluorescence:

Urogenital systems were fixed in 4% v/v paraformaldehyde/PBS, cryo-protected in 30% w/v sucrose/PBS, embedded in OCT compound, and sectioned, as described (Smith et al, 2008, supra). Immunofluorescence was used to assess protein expression. Rabbit DMRT1, anti-aromatase and anti-SOX9 (1:6000) were all raised in-house and have been described (Smith et al, 2003, supra).

### Example 1

### Down-Regulation of DMRT1

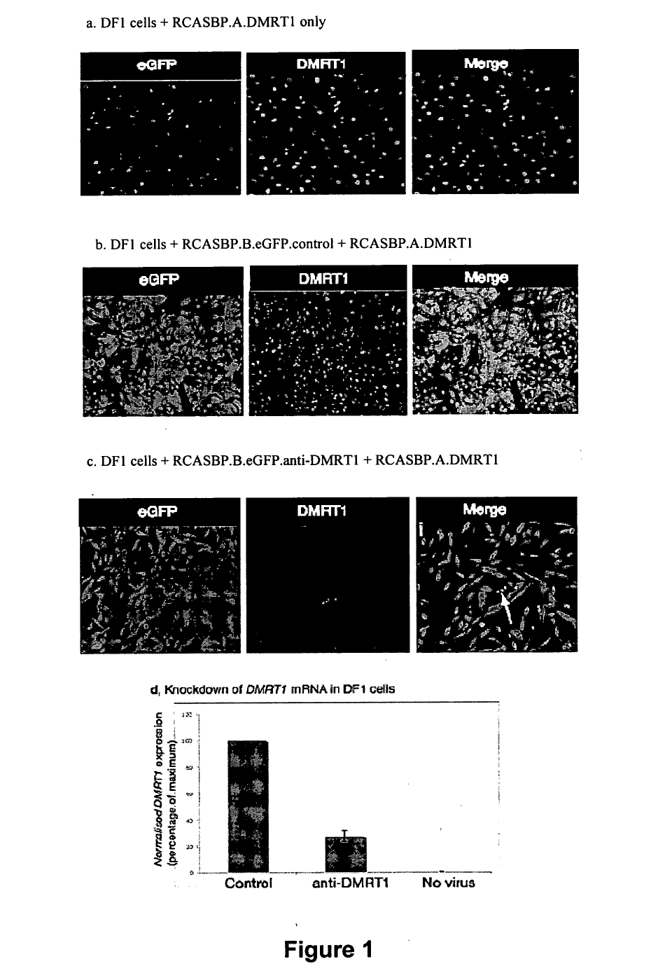

Recombinant microRNA (miRNA) directed against the DMRT1 transcript was delivered into living chicken embryos via the avian retroviral vector, RCASBP.B (Replication Competent Avian Sarcoma leukosis virus, high titre Bryan Polymerase, strain B). The virus was engineered to carry the open reading frame of GFP, to monitor viral spread, with the DMRT1 miRNA in the 3′ UTR of the transgene. The strong viral LTR promoter rather than an internal U6 promoter drove miRNA expression. This virus delivered robust GFP expression and knockdown of exogenous DMRT1 protein in cultured chicken DF1 cells (FIG. 1). Cells infected with RCASBP.A strain virus expressing only the DMRT1 cDNA showed strong DMRT1 over-expression (FIG. 1a). Cells co-infected with virus carrying DMRT1 cDNA and virus expressing a non-silencing RCASBP.B.GFP.scrambled miRNA showed robust DMRT1 protein expression (FIG. 1b). In contrast, cells co-infected with DMRT1 and the DMRT1 miRNA sequence showed knockdown of DMRT1 protein (FIG. 1c), and a 70% reduction in DMRT1 transcript compared to cells treated with scrambled control miRNA (FIG. 1d).

### Example 2

### Generation of Genetically Modified Embryos

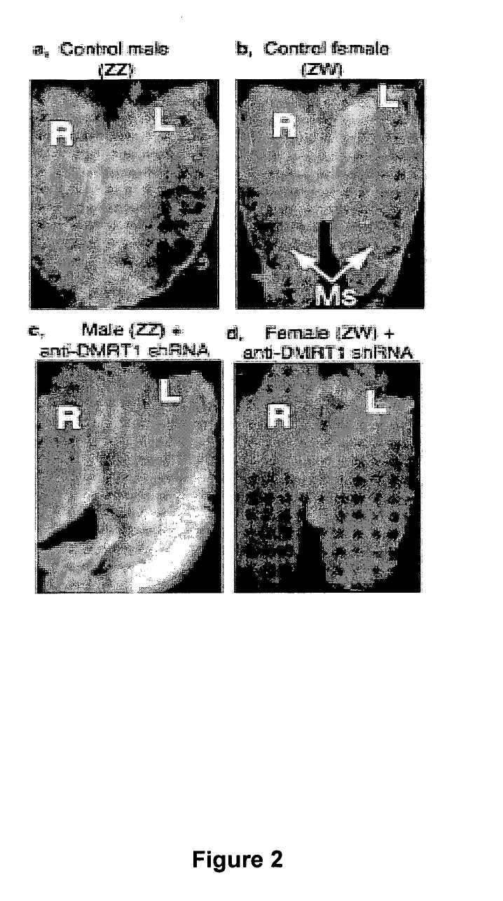

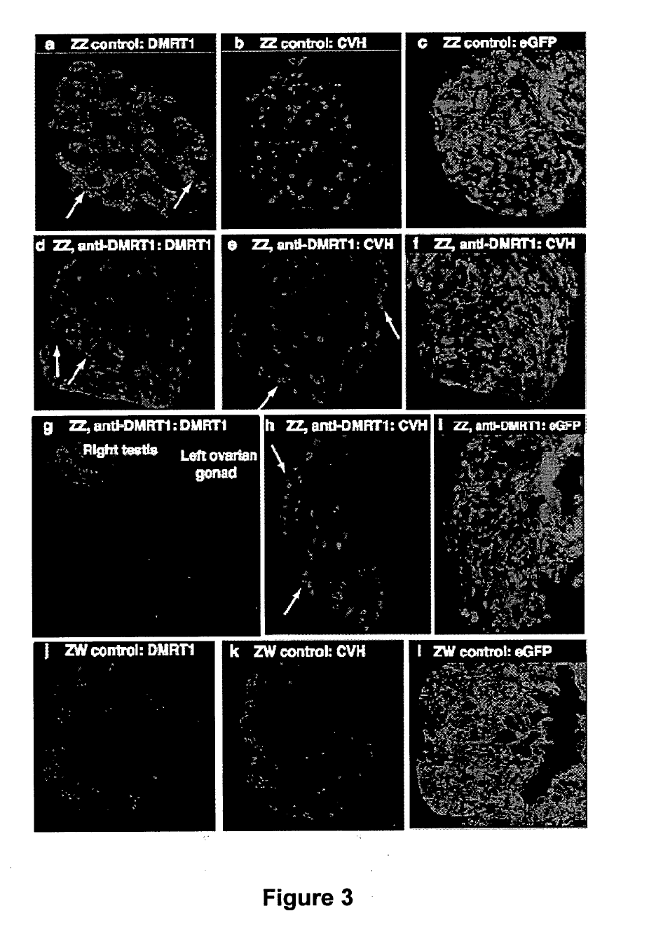

Virus carrying the GFP reporter together with DMRT1 miRNA was used to infect day zero chicken blastoderms, and embryogenesis was allowed to proceed until day 10. Control embryos were infected with virus carrying GFP and the scrambled non-silencing miRNA sequence. All embryos were genotypically sexed by PCR amplification of a W (female)-specific Xho1 repeat sequence. In the chicken embryo, the gonads form on the mesonephric kidneys around day 3.5 of incubation. Sexual differentiation into testes or ovaries begins at day 6 and is normally advanced by day 10. Embryos infected with virus at day zero showed robust global expression of GFP by day 10, including widespread expression in the urogenital system and in sectioned 25 gonads. Overall embryonic development was normal. Gonadal development in embryos treated with control miRNA was normal, with bilateral testes in males and typical asymmetric ovarian development in females (FIGS. 2a and b) [n=10]. In all control cases, gonadal sex matched genotypic sex. However, among those embryos treated with DMRT1 miRNA, five males that showed high GFP expression also showed disrupted testicular development. Macroscopically, three of these males showed abnormal (female-like) asymmetry (larger left and smaller right gonads) [FIG. 2c]. Females (ZW) treated with DMRT1 miRNA showed normal asymmetric ovarian development (FIG. 2d) [n=5]. Gonads from embryos with high levels of GFP (and hence miRNA delivery) were sectioned and assessed for DMRT1 and marker gene expression. In control embryos infected with virus carrying the non-silencing scrambled miRNA, DMRT1 expression was not affected and gonadal histology was normal. In control males, DMRT1 protein was uniformly expressed in developing Sertoli and germ cells of testis cords (FIG. 3a). Expression was strong, bilateral (in both left and right gonads) and indistinguishable from staining in uninfected male embryos. Germ cells were distributed 10 within testis cords in the medulla of both gonads, as assessed by staining for Chicken Vasa homologue (CVH) [FIG. 3b]. GFP immunofluorescence confirmed widespread expression of the scrambled miRNA sequence in control males (FIG. 3c).

In contrast, five male embryos (ZZ) treated with DMRT1 miRNA showed variably reduced DMRT1 protein expression in the left gonad, disrupted testis cord formation and ectopic female gene expression. The extent of DMRT1 knockdown and testis cord disruption varied among embryos. Some individuals showed disrupted DMRT1 expression, with disorganized cords and a cortical (female-like) pattern of germ cell distribution (FIGS. 3d and e). As in control males, these individuals showed strong GFP expression (FIG. 30. Other ZZ embryos showed stronger feminization, characterized by normal DMRT1 expression and cord formation in the right gonad, but greatly reduced DMRT1 expression, loss of cord organization and ovarian-type left gonad (FIG. 3g). The germ cells of the left feminized male gonads again exhibited a female-like distribution (i.e. concentrated in the outer part of the gonad, the cortex, rather than within testis cords) [FIG. 3h]. Female-type morphology of ZZ male gonads treated with DMRT1 miRNA was also revealed by fibronectin immunofluorescence, which delineated the presence of a thickened ovarian-like cortex and poorly formed cords. The strongest examples of ZZ feminization were observed in those gonads showing the strongest GFP expression (i.e. the highest delivery of the knockdown 30 miRNA) [FIGS. 3i and l].

In control females (ZW), ovarian development was normal. DMRT1 was lowly expressed in the developing (left) ovary, with the exception of higher expression in cortical germ cells (FIG. 3j). CVH staining revealed normal cortical germ cell distribution in controls (FIG. 3k). As in males, GFP expression was widespread in control females (FIG. 3l). In genetic females treated with DMRT1 microRNA, endogenous DMRT1 expression appeared lower, but the gonads nevertheless appeared normal, with typical asymmetry. This indicates that DMRT1 is not essential for chicken ovarian development.

### Example 3

### Characterization of Embryos

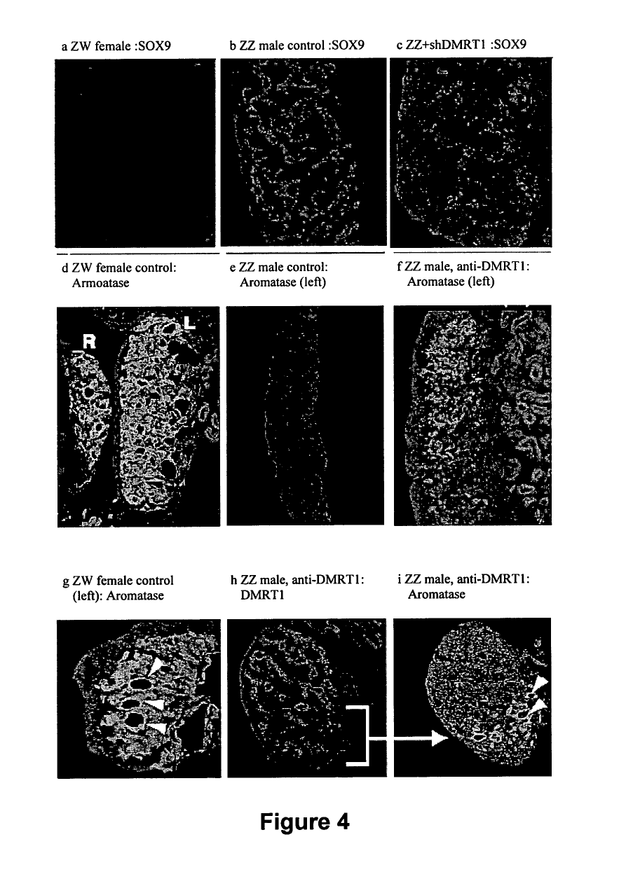

Gonads were further examined for the expression of male and female markers. A key gene involved in testicular differentiation is SOX9. In mammals, SOX9 is activated by SRY and is required for Sertoli cell differentiation and proper testis development. SOX9 is male up-regulated in all vertebrates that have been examined, including birds, pointing to a conserved role in testicular development. In day 10 control embryos infected with scrambled miRNA, SOX9 was expressed normally in male gonads. Female gonads lacked SOX9 expression, as expected (FIGS. 4a and b). In genetic males (ZZ) treated with DMRT1 miRNA, SOX9 protein expression was variably reduced, reflecting disrupted testis cords (FIG. 4c). DMRT1 may therefore play a role in the activation or maintenance of SOX9 expression during testis determination in the chicken embryo (a role supplanted by SRY in mammals). Genetic males treated with DMRT1 miRNA also showed ectopic activation of the robust female marker, aromatase. Aromatase enzyme is normally expressed only in female gonads, where it synthesizes the oestrogen that is required for ovarian differentiation in the chicken. Aromatase enzyme is never detected in normal male embryonic gonads. In control and knockdown female (ZW) embryos, aromatase was expressed normally in the medulla of both left and right gonads (FIG. 4d). No expression was seen in male controls (FIG. 4e). However, in the five genetic males feminized with DMRT1 miRNA, aromatase was activated in the left (but not right) gonad (FIG. 4f). In some genetic males (ZZ) with partial knockdown, areas of reduced or absent DMRT1 expression correlated with ectopic aromatase expression and female-like lacunae (FIGS. 4g, h, i). These findings suggest that elevated DMRT1 in male gonads normally suppresses aromatase and hence female development. This effect could be direct, or indirect via repression of the 5 FOXL2 gene, which is thought to activate aromatase.

### Example 4

### Mechanism of Action

The results of research underlying the invention support the Z dosage hypothesis for 10 avian sex determination (Nanda et al, Cytogenet Genome Res 122:150-156, 2008). A higher dosage of DMRT1 initiates testicular differentiation in male embryos, activating SOX9 expression and suppressing aromatase, which is essential for female development. DMRT1 fulfils the requirements expected of an avian master sex-determining gene. It is sex-linked, conserved on the Z sex chromosome of all birds examined, including the basal ratites (ostriches, emus, etc) [Sheety et al, cytogenet Genome Res 99:245-251, 2002]. It is expressed exclusively in the urogenital system prior to gonadal sex differentiation in chicken embryos, with higher expression in males (Smith et al, 2003, supra), and knockdown leads to gonadal sex reversal. In the other vertebrates, DMRT1 is also implicated in testis determination. In reptiles with temperature sex determination, DMRT1 expression is up-regulated during the thermosensitive period when sex is being determined, and only at male-producing temperatures (Shoemarker et al, Dev Dyn 236:12055-1063, 2007; Kettlewell et al, Genesis 26:174-178, 2000). In the medaka fish, Oryzias latipes, a duplicated copy of DMRT1, DMY, is the master testis determinant (Matsuda et al, Nature 417:559-563, 2002), while a W-linked copy, DMW, is involved in ovarian development in an amphibian, Xenopus laevis (Yoshimoto et al, Proc Natl Acad Sci USA 105:2469-2474, 2008). The data herein provide evidence that DMRT1 is the elusive male sex determinant in birds.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Arit et al, Proc. Biol. Sci. 271 Suppl. 4:S249-251, 2004
Ellegren, Trends Ecol Evol 15:188-192, 2000
Kettlewell et al, Genesis 26:174-178, 2000
Matsuda et al, Nature 417:559-563, 2002
Mizuno et al, Cytogenet Genome Res 99:236-244, 2002
Nakagawa, Trends Genet 20:479-480, 2004
Nanda et al, Cytogenet Genome Res 122:150-156, 2008
Shan et al, Cytogenetics Cell Genetics 89:252-257, 2000
Sheety et al, cytogenet Genome Res 99:245-251, 2002
Shoemarker et al, Dev Dyn 236:12055-1063, 2007
Smith et al, Nature 402:601-602, 1999
Smith et al, Biol Reprod 68:560-567, 2003
Smith et al, BMC Dev Biol 8:72, 2008
Smith et al, Int. J. Dev. Biol. 53:59-67, 2009
Smith et al, Nature Letters 46:267-271, 2009
Smith and Sinclair, Bioassays 26:120-132, 2004
Yoshimoto et al, Proc Natl Acad Sci USA 105:2469-2474, 2008

1-26. (canceled)

27. A sex specified animal, said animal or its parent genetically modified to either (i) inhibit expression of DMRTI or a male chromosome-linked homolog thereof or reduce activity of DMRTI protein or its homolog; or (ii) to elevate expression of an exogenous DMRTI or a male chromosome-linked homolog thereof or activity of DMRTI protein or its homolog wherein reduced expression of DMRTI or DMRTI protein activity or of its homolog leads to an animal with female characteristics and elevated expression of DMRTI or DMRTI protein activity or of its homolog leads to an animal with male characteristics.

28. The sex-specified animal of claim 27 wherein expression of DMRTI or activity of DMRTI protein or of its homolog is reduced leading to an animal with female characteristics, or expression of DMRTI or DMRTI protein activity or of its homolog is elevated leading to an animal with male characteristics.

29. The sex-specified animal of claim 27 selected from the group consisting of an avian animal, a reptile, a fish and an amphibian.

30. The sex-specified animal of claim 29 wherein the animal is an avian animal and the avian animal is selected from the group consisting of a chicken, duck, goose, turkey, bantam, pheasant and a quail.

31. The sex-specified animal of claim 29 wherein the animal is an avian animal and the avian animal is a chicken.

32. The sex-specified animal of claim 27 wherein the reduced level of DMRTI expression is due to a genetic modification which targets DMRTI or its genetic locus.

33. The sex-specified animal of claim 28 wherein the animal is an avian animal and the avian animal expresses an exogenous DMRTI gene or its homolog.

34. Progeny of the sex specified animal of claim 27.

35. A method for generating a sex-specified animal, said method comprising introducing into a blastoderm or developing embryo of the animal an agent which modulates the level of expression of DMRTI or a male chromosome-linked homolog thereof or modulates the activity of DMRTI protein and allowing the embryo to develop into a postnatal animal wherein an agent which reduces expression of DMRTI or DMRTI protein activity results in an animal which elevates expression of DMRTI or DMRTI protein activity results in an animal with male characteristics.

36. The method of claim 35 wherein the animal is an avian animal.

37. A method for generating a sex-specified avian animal, said method comprising introducing into a blastoderm or developing embryo of the avian animal an agent which modulates the level of expression of DMRTI or its homolog or the level of activity of DMRTI protein.

38. A method of inducing feminization of an avian embryo, the method comprising introducing to the embryo an agent which reduces the functional level of DMRTI expression or DMRTI protein for a time and under conditions sufficient for the embryo to develop female characteristics.

39. A method of inducing masculinization of an avian embryo, the method comprising introducing to the embryo an agent which comprises DMRTI protein or its functional homolog or analog or which facilitates expression of DMRTI protein or its functional homolog for a time and under conditions sufficient for the embryo to develop male characteristics.

40. A method for generating a female avian animal, said method comprising genetically modifying an avian embryo in ovo to inactivate expression of DMRTI or its homolog for a time and under conditions sufficient for the embryo to be feminized and allowing the feminized embryo to mature and hatch.

41. A sex-specified non-human animal wherein the sex of the animal is determined by the dose (x) of a male chromosome (M)-linked sex regulatory gene and wherein a homogametic (MM) male has a 2x dose of the regulatory gene and a heterogametic (MF) female, wherein F is the female gamete, has a lx dose of the regulatory gene, the gender specified animal or its parent genetically modified by a means selected from the group consisting of:
(i) down-regulating expression of one or both alleles of the sex regulatory gene on the M chromosome to a dose of <2x, wherein the gender is female;
(ii) up-regulating expression of the sex regulatory gene on a M chromosome in a MF female to a dose level of 2x or more wherein the gender is male; and
(iii) introducing and expressing an exogenous sex regulatory gene to the F or M chromosome, wherein the gender is male;
wherein the sex regulatory gene is DMRTI or a male chromosome-linked homolog thereof.

42. The sex-specified non-human animal of claim 41 wherein the animal is an avian animal.

43. The sex-specified non-human animal of claim 42 wherein the animal is a female.

44. The sex-specified non-human animal of claim 43 wherein the dose of the sex regulatory gene is substantially zero.

45. An agent which inhibits DMRTI gene expression or DMRTI protein activity in the manufacture of a feminized avian animal, or an agent which facilitates DMRTI expression or protein activity in the manufacture of a masculinized avian animal.

46. A business model provided for the generation of female poultry birds with enhanced economic returns, the model comprising generating female poultry birds with an inactivated DMRTI gene or gene locus, mating these birds to one or more male birds to generate fertilized eggs, allowing the eggs to hatch wherein the resulting hatchlings are all female and wherein the female birds are reared and introduced in an existing poultry bird operation.


SEQUENCE LISTING (4 sequences)

SEQ ID NO: 1 - DNA, 20 bp, Artificial
  DMRT1 probe (forward)
agcctcccagcaacatacat

SEQ ID NO: 2 - DNA, 18 bp, Artificial
  DMRT1 probe (reverse)
gcggttctccatcccttt

SEQ ID NO: 3 - DNA, 21 bp, Artificial
  HPRT probe (forward)
cgccctcgactacaatgaata

SEQ ID NO: 4 - DNA, 21 bp, Artificial
  HPRT probe (reverse)
caactgtgctttcatgctttg